(19) United States
(12) Plant Patent Application Publication
(10) Pub. No.: US 2008/0184425 P1
(43) Pub. Date: Jul. 31, 2008

(54) Wahlenbergia plant named 'SPICI BELL'

(75) Inventor: Finn Plougheld, Odense (DK)

Correspondence Address: FOLEY AND LARDNER LLP;SUITE 500, 3000 K STREET NW, WASHINGTON, DC, 20007, US

(73) Assignee: GARTNERIET LUNDEGAARD APS

(21) Appl. No.: 11/651,509

(22) Filed: Jan. 10, 2007

(51) Int. Cl. A01H 5/00

(52) U.S. Cl. PLT/263.1

(57) ABSTRACT

A new and distinct cultivar of Wahlenbergia plant named ‘SPICI BELL’, characterized by having globular, compact plant habit; dense and bushy plant form, mainly due to a higher quantity of shorter stems; vigorous growth habit, but less need for chemical growth retardation; and higher number of upright, white flowers per plant.

### LATIN NAME OF GENUS AND SPECIES OF THE PLANT CLAIMED

Wahlenbergia procumbens

### VARIETY DENOMINATION

‘SPICI BELL’

### BACKGROUND OF THE INVENTION

The present invention relates to a new and distinct cultivar of Wahlenbergia plant, botanically known as Wahlenbergia procumbens Thunb., commonly known as Wild Violet, and hereinafter referred to by the name ‘SPICI BELL’.

The new Wahlenbergia, ‘SPICI BELL’, is a product of a planned breeding and selection and monitoring program conducted by the inventor, Finn PLOUGHELD, in Korup, Denmark. The new Wahlenbergia cultivar originated from a polycross among seed plants of wild type Wahlenbergia procumbens and was selected in 2004 by the inventor. The inventor selected the new Wahlenbergia cultivar on the basis of its sturdy, compact and freely flowering habit. Plants of the new Wahlenbergia are more globular, compact and more freely flowering than plants of the wild type.

Asexual reproduction of the new Wahlenbergia cultivar by terminal cuttings taken and propagated since March of 2004 in Korup, Denmark, has shown that the combination of characteristics as herein disclosed for the new cultivar are firmly fixed and retained through successive generations of asexual reproduction. The new cultivar reproduces true-to-type.

### SUMMARY OF THE INVENTION

The following traits have been repeatedly observed and are determined to be the unique characteristics of ‘SPICI BELL’. These characteristics in combination distinguish ‘SPICI BELL’ as a new and distinct cultivar:

1. Globular, compact plant habit;

2. Dense and bushy plant form, mainly due to a higher quantity of shorter stems;

3. Vigorous growth habit, but less need for chemical growth retardation; and

4. Higher number of upright, white flowers per plant.

Side-by-side comparisons between plants of the new Wahlenbergia cultivar, ‘SPICI BELL’ and plants of the Wahlenbergia procumbens wild type were conducted by the inventor in Korup, Denmark. Plants of ‘SPICI BELL’ differ from plants of the Wahlenbergia procumbens wild type in the following characteristics:

1. Plants of ‘SPICI BELL’ have pure white, upright, campanulate flowers.

2. Plants of ‘SPICI BELL’ have shorter internodes and petioles, but larger leaves and more compact, globular growth than plants of the Wahlenbergia procumbens wild type.

3. Plants of ‘SPICI BELL’ have shorter flower peduncles than plants of the Wahlenbergia procumbens wild type.

4. Plants of ‘SPICI BELL’ are more compact than the plants of the Wahlenbergia procumbens wild type.

5. Plants of ‘SPICI BELL; have more flowers per plant than the plants of the Wahlenbergia procumbens wild type.

### BRIEF DESCRIPTION OF THE PHOTOGRAPHS

The accompanying colored photographs illustrate the overall appearance of the new cultivar ‘SPICI BELL’, showing the colors as true as it is reasonably possible to obtain in colored reproductions of this type. Colors in the photographs may differ slightly from the color values cited in the detailed botanical description, which more accurately describe the actual colors of ‘SPICI BELL’.

FIG. 1 shows a side perspective view of a typical flowering plant of ‘SPICI BELL’, as grown in a 9.0 cm pot.

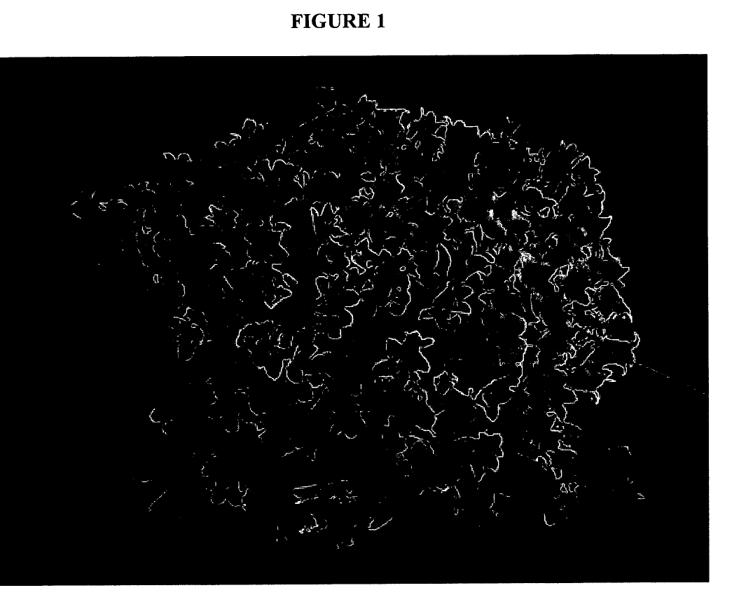

FIG. 2 shows a close-up view of typical flowering racemes, buds and leaves of‘SPICI BELL’.

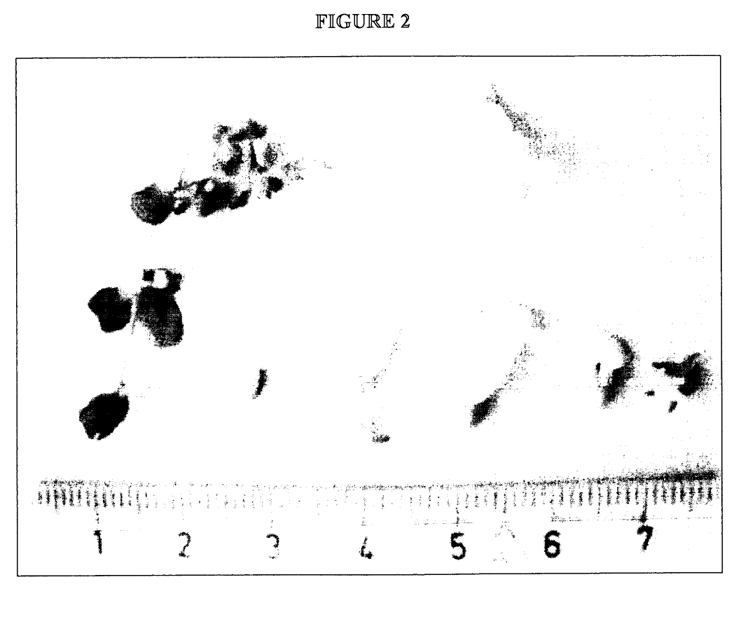

FIG. 3 shows a side perspective view of a typical flowering plant of the Wahlenbergia procumbens wild type, as grown in a 9.0 cm pot.

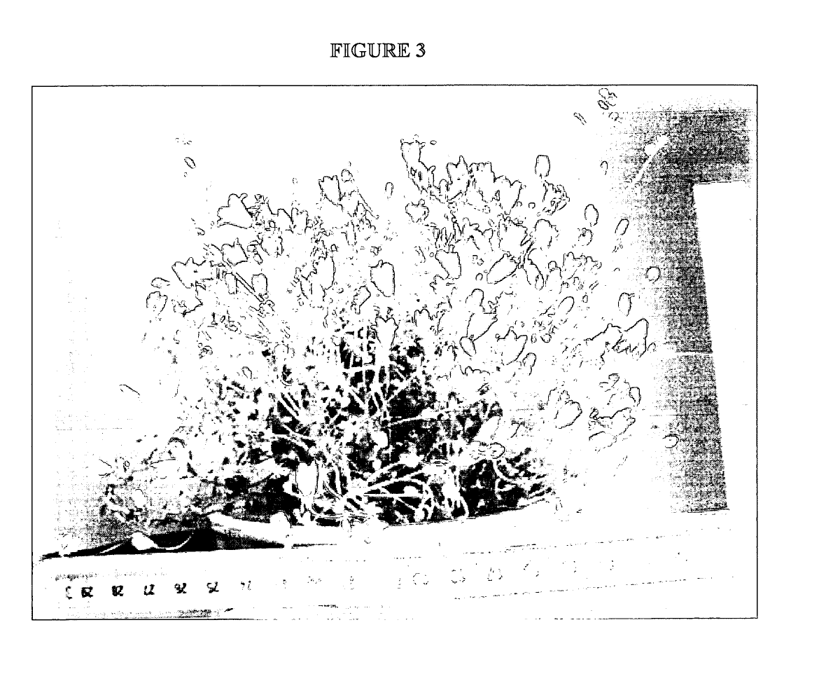

FIG. 4 shows a close-up view of typical flowering racemes, buds and leaves of the Wahlenbergia procumbens wild type.

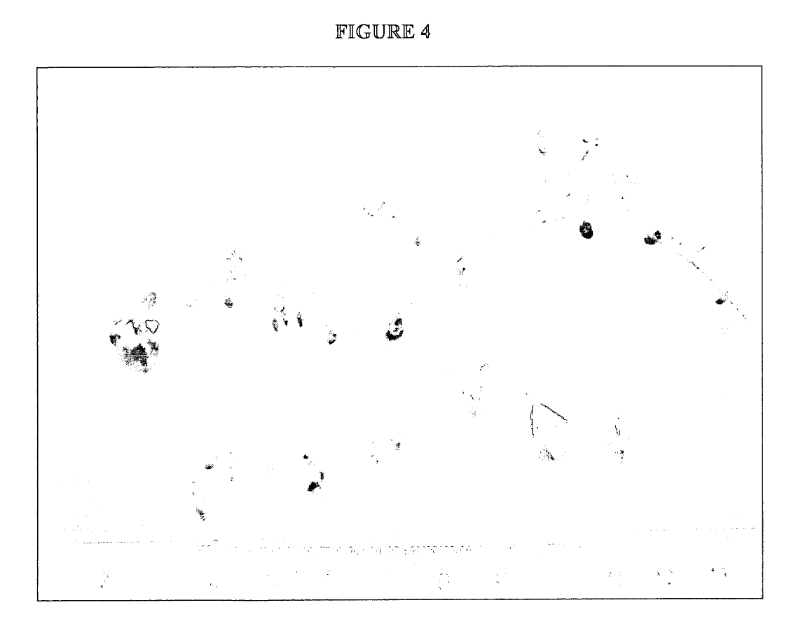

### DETAILED BOTANICAL DESCRIPTION

The new Wahlenbergia cultivar ‘SPICI BELL’ has not been observed under all possible environmental conditions. The phenotype of the new cultivar may vary with variations in environment such as temperature, light intensity, day length, and fertility level without any variance in genotype.

The aforementioned photographs, together with the following observations, measurements and values describe plants of the new Wahlenbergia cultivar ‘SPICI BELL’ as grown in a heated and lighted, glass-covered greenhouse in Korup, Denmark, under conditions which closely approximate those generally used in commercial practice. The day temperatures in the greenhouse ranged from 20-30° C. and the night temperatures ranged from 17-25° C. Ambient light levels were used while growing plants of ‘SPICI BELL’. No photoperiodic treatments were used when growing plants of ‘SPICI BELL’. The age of the ‘SPICI BELL’ plants described is 14 weeks old (after cutting, and retarded once using Paclobutrazol) and grown in 9.0 cm pots. For commercial production, some retardation will be required, but less that is required for the parent wild type.

The photographs and descriptions were taken during the summer-fall season when day temperatures in glass-covered greenhouse range from 20-30° C. and when night average temperatures in glass-covered greenhouse range from 17-25° C.

Color references are made to The Royal Horticultural Society Color Chart (RHS), 4^{th} edition, except where general colors of ordinary significance are used.
Classification:
Botanical: Wahlenbergia procumbens
Parentage: Seed plant from polycross of Wahlenbergia procumbens wild type
Propagation:
Type cutting: Terminal vegetative cuttings. Two or more per pot depending on pot size.
Time to initiate roots: About 10 to 14 days at 18 to 21° C., with transparent foil covering, in a greenhouse.
Root description: Fine, well branched
Plant description:
Form: Perennial plant with upright, spreading, globular plant habit. Campanulate flowers in racemes. Freely branching with lateral flowering branches forming at every node.
Crop time: After rooting, about 11 weeks are required to produce finished flowering plants in 9.0 cm pots.
Vigor: Vigorous growth rate
Plant height (soil level to top of plant plane): About 2.5 cm
Plant spread (width): About 16 cm
Lateral branches: Quantity: About 200 per plant. Length: About 6 cm. Diameter: About 0.5 mm. About 10 to 20 leaves per lateral branch.
Internode length: 3.0 mm to 5.0 mm.
Stem: Shape: Round. Strength: Soft. Aspect: Creeping. Pubescence: None. Color: RHS 138B, green.
Foliage description: Single leaves arranged basally opposite and apically alternate. If foliage dried, curry fragrance.
   Length.—Length: About 4 mm.
   Width.—About 5 mm.
   Overall shape.—Round to obovate.
   Apex.—Obtuse.
   Base.—Cuneate, sessile.
   Margin.—Entire.
   Texture.—Glabrous.
   Pubescence.—None.
   Color (immature).—Upper side: RHS 137C, green Underside: RHS 138B, green.
   Color (mature).—Upper side: RHS 137C, green Underside: RHS 138B, green.
   Venation.—Color: Upper surface: RHS 137C, green. Upper surface: RHS 138D, green. Pattern: Costate.
Flower description:
Flower arrangement and shape: Upright campanulate flowers in racemes. Flowers with small star shaped calyx.
Natural flowering season: Continuous throughout the summer and fall. Season can be extended by exposure to high irradiance.
Flower longevity: Longevity of individual flowers on plant is about 4-5 weeks. Longevity of individual flowers is highly dependent on temperature and light conditions. Flowers persistent, but wither to insignificance.
Time to flower: About 4-5 weeks.
Fragrance: Weak.
Inflorescence size: About 5-6 cm.
Number of flowers per inflorescence: About 10
Number of flowers per lateral stem: 5
Number of flower buds per lateral stem: 4
Number of flowers and buds per plant: About 400
Buds:
   Length.—Up to 9 mm.
   Diameter.—Up to 3 mm.
   Shape.—Clavate.
   Color.—RHS 149D, light yellow green.
Petals:
   Quantity.—About 5.
   Length.—About 12 mm.
   Diameter.—About 15 mm.
   Shape.—Triangular, acute petal lobes, which are 3-6 mm long and 4-6 mm wide at base. Fused.
   Appearance.—Glabrous.
   Texture.—Almost translucent, bi-ridged.
   Margin.—Entire.
   Color (mature).—Both surfaces: White, whiter than RHS 155A.
   Color (immature).—Both surfaces: White, whiter than RHS 155A.
   Color Fades.—RHS 161A,greyed-yellow.
Sepals:
   Quantity per flower.—5, occasionally 6.
   Arrangement.—Free.
   Appearance.—Star shaped.
   Length.—About 3.0 mm.
   Width.—Basally: about 1.5 mm.
   Overall shape.—Lanceolate.
   Tip.—Aristulate.
   Base.—Sessile, fused to ovary.
   Margin.—Entire.
   Texture.—Smooth.
   Color (immature).—Upper and under surfaces, RHS 145B, yellow-green.
   Color (mature).—Upper surface: RHS 143B, green. Under surface: RHS 143A, green.
Peduncles:
   Length.—About 6.0 mm to 11.0 mm.
   Diameter.—About 0.5 mm.
   Strength.—Moderately Strong.
   Angle.—Varies from 15 to 30 degrees from stem.
   Color.—RHS 138C, green.
Reproductive organs:
Androecium:
   Stamen.—Quantity: About 5.
   Anther.—Shape: Clavate. Length: About 2.5 mm. Color: RHS 155B, white.
   Pollen.—Amount: Moderate. Color: RHS 155C, white.
Gynoecium:
   Pistil.—Quantity: 1. Length: About 4.5 mm.
   Stigma.—Shape: Tripartite. Color: RHS 155C, white.
   Style.—Length: About 4 mm. Color: RHS 155A, white.
   Ovary.—Color: RHS 155A, white.
Seeds:
   Quantity.—About 100 per flower.
   Length.—About 1.0 mm.
   Diameter.—About 1.0 mm.
   Texture.—Glabrous, hard.
   Color.—RHS 199A, grey-brown.
Growth retardants: Paclobutrazol and STS
Weather tolerance: Plants of the new Wahlenbergia have exhibited good tolerance to drought, rain and wind, with low temperature resistance to +1.5° C.
Disease/pest resistance: ‘SPICI BELL’ is not resistant to any diseases or pests typical to Wahlenbergia cultivars.
Disease/pest susceptibility: ‘SPICI BELL’ is susceptible to Botrytis.

I claim:

1. A new and distinct cultivar of Wahlenbergia plant named ‘SPICI BELL’, as illustrated and described herein.
